(19) United States
(12) Patent Application Publication
(10) Pub. No.: US 2015/0031991 A1
(43) Pub. Date: Jan. 29, 2015

(54) Depth Advancement Marker Needle For Image Guided Procedures

(71) Applicant: John Claude Elfar, Rochester, NY (US)

(72) Inventor: John Claude Elfar, Rochester, NY (US)

(73) Assignee: UNIVERSITY OF ROCHESTER, Rochester, NY (US)

(21) Appl. No.: 14/445,427

(22) Filed: Jul. 29, 2014

Related U.S. Application Data
(60) Provisional application No. 61/859,657, filed on Jul. 29, 2013.

(51) Int. Cl. A61B 5/06; A61M 5/46

(52) U.S. Cl. CPC A61B 5/066; A61M 5/46
USPC 600/424; 604/117

(57) ABSTRACT

Many image guided needle procedures use ultrasound, computed tomography (CT) magnetic resonance imaging (MRI) or other imaging systems. In such procedures, there is currently a preference for long axis injections and related procedures where the long axis of the needle is parallel to the plane of the two dimensional images created by the imaging system. This is due to the fact that long axis imaging of a needle provides good visual indication of depth of penetration of the needle, whereas short axis imaging shows only a dot or small circle, which is the cross section of the needle and provides no depth of penetration information. Many procedures, however, benefit from, or require the use of, short axis imaging.

The depth advancement marker needle of the present invention provides a feature along the shaft of the needle that provides a visual indicator of depth of penetration while performing short axis imaging.

### CROSS REFERENCE TO RELATED PATENT APPLICATIONS

This application claims priority to U.S. Patent Application Ser. No. 61/859,657 filed Jul. 29, 2013 entitled “Depth Advancement Marker Needle For Image Guided Injections” by John Claude Elfar of Rochester, N.Y. the entire disclosure of which is incorporated herein by reference.

### BACKGROUND OF THE INVENTION

1. Field of the Invention

The present invention relates generally to medical needles, and more particularly to a medical needle that provides depth of penetration information while using short axis imaging techniques.

2. Description of the Related Art

Image guided needle procedures are common practice today. Many of these procedures use ultrasound, computed tomography (CT), Magnetic Resonance imaging (MRI) or other imaging techniques where images are taken as “slices” of specific areas of the body. Such images make it difficult to determine depth of penetration of a needle or similar surgical instrument in the so-called “short axis” where the long axis of the needle is perpendicular to the two dimensional image (“slice”). While the needle can be seen in such a short axis image, it appears as a dot or small circle and the depth of penetration cannot readily be determined. Thus, there is currently a preference for long axis injections and related procedures where the long axis of the needle is parallel to the plane of the two dimensional images (“slice”). This preference is fully justified, as depth of penetration is critical information for many image guided needle procedures. The preference for long axis injections due to imaging concerns is often a compromise, as there are many procedures that would benefit from a short axis image guided procedure were it not for the concern over lack of depth of penetration information.

What is therefore needed and beneficial is a medical needle that provides depth of penetration information when short axis imaging is performed.

It is thus an object of the present invention to provide a medical needle that provides depth of penetration information while using short axis imaging techniques. It is another object of the present invention to provide a medical needle that provides depth attainment information while using short axis imaging techniques. It is yet another object of the present invention to provide a medical needle that facilitates penetration while providing depth of penetration information. It is a further object of the present invention to provide a method of determining depth or distance of penetration while using a medical needle of the present invention. These and other objects of the present invention are not to be considered comprehensive or exhaustive, but rather, exemplary of objects that may be ascertained after reading this specification with the accompanying drawings and claims.

### BRIEF SUMMARY OF THE INVENTION

In accordance with the present invention, there is provided a depth advancement marker needle for image guided procedures comprising a hollow metal wire having a shaft, a sharp end and a fastening end; and a feature that provides a visual indicator of depth of penetration while performing short axis imaging.

The foregoing paragraph has been provided by way of introduction, and is not intended to limit the scope of the invention as described by this specification and the attached drawings and claims.

### BRIEF DESCRIPTION OF THE DRAWINGS

The invention will be described by reference to the following drawings, in which like numerals refer to like elements, and in which:

FIG. 1 depicts a perspective view of an ultrasonic transducer probe performing short axis imaging of an image guided injection;

FIG. 2 is a plan view of the imaging procedure of FIG. 1;

FIG. 3 is a perspective view of a first embodiment of a depth advancement marker needle;

FIG. 4 is a plan view of a first embodiment of the depth advancement marker needle;

FIG. 5 is an end view of a first embodiment of a depth advancement marker needle;

FIG. 6 is a cross sectional view of the depth advancement marker needle taken along line N-N of FIG. 4;

FIG. 7 is a cross sectional view of the depth advancement marker needle taken along line M-M of FIG. 4;

FIG. 8 is a cross sectional view of the depth advancement marker needle taken along line A-A of FIG. 5;

FIG. 9 is a perspective view of a second embodiment of a depth advancement marker needle;

FIG. 10 is a plan view of a second embodiment of the depth advancement marker needle;

FIG. 11 is an end view of a first embodiment of a depth advancement marker needle;

FIG. 12 is a cross sectional view of the depth advancement marker needle taken along line R-R of FIG. 10;

FIG. 13 is a cross sectional view of the depth advancement marker needle taken along line T-T of FIG. 10;

FIG. 14 is a cross sectional view of the depth advancement marker needle taken along line P-P of FIG. 11;

FIG. 15 is a perspective view of a third embodiment of a depth advancement marker needle;

FIG. 16 is a plan view of a third embodiment of the depth advancement marker needle;

FIG. 17 is an end view of a third embodiment of a depth advancement marker needle;

FIG. 18 is a close-up detail view of the depth advancement marker needle of FIG. 16;

FIG. 19 is a cross sectional view of the depth advancement marker needle taken along line B-B of FIG. 17;

FIG. 20 is a perspective view of a fourth embodiment of a depth advancement marker needle;

FIG. 21 is a plan view of a fourth embodiment of the depth advancement marker needle;

FIG. 22 is an end view of a fourth embodiment of a depth advancement marker needle;

FIG. 23 is a cross sectional view of the depth advancement marker needle taken along line C-C of FIG. 22;

FIG. 24 is a close-up detail view of the depth advancement marker needle of FIG. 23;

FIG. 25 is a perspective view of a fifth embodiment of a depth advancement marker needle;

FIG. 26 is a plan view of a fifth embodiment of the depth advancement marker needle;

FIG. 27 is an end view of a fifth embodiment of a depth advancement marker needle;

FIG. 28 is a cross sectional view of the depth advancement marker needle taken along line U-U of FIG. 27;

FIG. 29 is a close-up detail view of the depth advancement marker needle of FIG. 28;

FIG. 30 is a perspective view of a sixth embodiment of a depth advancement marker needle;

FIG. 31 is a plan view of a sixth embodiment of the depth advancement marker needle;

FIG. 32 is an end view of a sixth embodiment of a depth advancement marker needle;

FIG. 33 is a cross sectional view of the depth advancement marker needle taken along line V-V of FIG. 32;

FIG. 34 is a close-up detail view of the depth advancement marker needle of FIG. 33;

FIG. 35 is a perspective view of a seventh embodiment of a depth advancement marker needle;

FIG. 36 is a plan view of a seventh embodiment of the depth advancement marker needle;

FIG. 37 is a close-up detail view of the depth advancement marker needle of FIG. 36;

FIG. 38 is a cross sectional view of the depth advancement marker needle taken along line AA-AA of FIG. 37;

FIG. 39 is a cross sectional view of the depth advancement marker needle taken along line AB-AB of FIG. 37;

FIG. 40 is a cross sectional view of the depth advancement marker needle taken along line AC-AC of FIG. 37;

FIG. 41 is a cross sectional view of the depth advancement marker needle taken along line AD-AD of FIG. 37;

FIG. 42 is a cross sectional view of the depth advancement marker needle taken along line AE-AE of FIG. 37;

FIG. 43 is a cross sectional view of the depth advancement marker needle taken along line AF-AF of FIG. 37;

FIG. 44 is a cross sectional view of the depth advancement marker needle taken along line AG-AG of FIG. 37;

FIG. 45 is a cross sectional view of the depth advancement marker needle taken along line AH-AH of FIG. 37;

FIG. 46 is a cross sectional view of the depth advancement marker needle taken along line AI-AI of FIG. 37;

FIG. 47 is a cross sectional view of the depth advancement marker needle taken along line AJ-AJ of FIG. 37;

FIG. 48 is a perspective view of an eighth embodiment of a depth advancement marker needle;

FIG. 49 is a plan view of an eighth embodiment of the depth advancement marker needle;

FIG. 50 is an end view of an eighth embodiment of a depth advancement marker needle;

FIG. 51 is a close-up detail view of the depth advancement marker needle of FIG. 49;

FIG. 52 is a cross sectional view of the depth advancement marker needle taken along line BA-BA of FIG. 51;

FIG. 53 is a cross sectional view of the depth advancement marker needle taken along line BB-BB of FIG. 51;

FIG. 54 is a cross sectional view of the depth advancement marker needle taken along line BC-BC of FIG. 51;

FIG. 55 is a cross sectional view of the depth advancement marker needle taken along line BD-BD of FIG. 51;

FIG. 56 is a cross sectional view of the depth advancement marker needle taken along line BE-BE of FIG. 51;

FIG. 57 is a cross sectional view of the depth advancement marker needle taken along line BF-BF of FIG. 51;

FIG. 58 is a cross sectional view of the depth advancement marker needle taken along line BG-BG of FIG. 51;

FIG. 59 is a cross sectional view of the depth advancement marker needle taken along line BH-BH of FIG. 51;

FIG. 60 is a cross sectional view of the depth advancement marker needle taken along line BI-BI of FIG. 51;

FIG. 61 is a cross sectional view of the depth advancement marker needle taken along line BJ-BJ of FIG. 51;

FIG. 62 is a cross sectional view of the depth advancement marker needle taken along line W-W of FIG. 50;

FIG. 63 is a perspective view of a ninth embodiment of a depth advancement marker needle;

FIG. 64 is a plan view of a ninth embodiment of the depth advancement marker needle;

FIG. 65 is an end view of a ninth embodiment of a depth advancement marker needle;

FIG. 66 is a cross sectional view of the depth advancement marker needle taken along line X-X of FIG. 65;

FIG. 67 is an ultrasound long axis image of an image guided injection procedure; and

FIG. 68 is an ultrasound short axis image of an image guided injection procedure.

The present invention will be described in connection with a preferred embodiment, however, it will be understood that there is no intent to limit the invention to the embodiment described. On the contrary, the intent is to cover all alternatives, modifications, and equivalents as may be included within the spirit and scope of the invention as defined by this specification, drawings and claims provided herein.

### DESCRIPTION OF THE PREFERRED EMBODIMENTS

The Depth Advancement Marker Needle For Image Guided Procedures of the present invention may have various embodiments, some of which are described herein, and others of which may be inferred from or otherwise envisioned based on the disclosure contained herein. As used herein, the term depth refers to the distance that the marker needle of the present invention travels or has traveled in any axis or orientation.

A medical needle is constructed from a hollow metal wire having a shaft, a sharp end that is usually beveled, and a fastening end for connecting to a syringe or the like. Stainless steel is commonly used. The hollow metal wire, or cannula, is formed from a larger metal tube that is fabricated by rolling a sheet of metal into a tube and welding the resulting seam. Laser welding is commonly employed. Alternatively, a solid piece of metal may be bored and machined. Once this larger diameter metal tube is created, it is heated and drawn through a die to stretch the length of the tube while at the same time decrease the diameter of the tube. A series of progressively smaller dies are used to continue to decrease the diameter of the tube until the last die is used, typically without heat. This cold working of the tube increases the strength and hardness of the tube. Sometimes a mandrel of similar form is placed inside the tube to prevent tube wall collapse, but ordinarily the tolerances provided by the equipment and related manufacturing processes are such that a mandrel or form is not necessary. Once the hollow metal wire is created, it is cut to a specified length and a sharp end is created on a first end of the cut hollow metal wire by grinding, cutting a bevel, or the like. A fastener such as a LUER-LOCK® or a LUER-SLIP® connector is placed on the fastening end of the hollow metal wire by press fitting, friction fitting, adhesion, or the like. LUER-LOCK® and LUER-SLIP® are registered trademarks of Becton Dickinson and Company of Franklin Lakes, N.J.

The present invention and the various embodiments described and envisioned herein rely on the use of a feature along the shaft or other portion of the medical needle that provides a visual indicator of depth of penetration while performing short axis imaging. This feature may take on various embodiments, and can be seen with the assistance of an imaging system such as ultrasound, computed tomography (CT), Magnetic Resonance Imaging (MRI) or other imaging system, around the circumference of the medical needle either as a unique and readily identifiable feature, or as a feature that changes the overall diameter of the medical needle dependent on the depth of penetration of the medical needle. The feature may, in some embodiments, be a plurality of features the quantity of which relate to the depth of penetration of the medical needle during the image guided procedure.

The feature may be machined, stamped, chemical etched, laser etched, deposited, or otherwise placed on the sheet of metal prior to rolling or may, in some embodiments of the present invention, be machined, stamped, chemical etched, laser etched, deposited, or otherwise placed on the resulting tube either before, during, or after the die drawing process. In some embodiments of the present invention, the feature may be machined, stamped, chemical etched, laser etched, deposited, or otherwise placed on the resulting medical needle. The feature may also be created by variation on die drawing techniques, such as the creation of a varying diameter medical needle. The feature may be contained on the outer surface of the medical needle, or may, in some embodiments of the present invention, be contained on the inner surface of the medical needle.

Determination of the depth of penetration of a medical needle during an image guided procedure has important implications for procedural efficacy. In addition, most modern day imaging systems provide a digital record of the procedure, thus providing the clinician with a record of the depth of penetration for future diagnostics, medical liability, and the like. For example, confirmation of proper depth of penetration and a related record thereof will prove to be extremely valuable for radiation therapy to ensure that the therapeutic material was delivered to the proper depth and location at the tumor site. For biopsies, confirmation that the proper depth and location was reached will be of immense value to ensure that the correct tissue has been removed for the biopsy.

In some embodiments of the present invention, the depth advancement marker needle may be used for various localization procedures such as, for example, radiofrequency ablation, ultrasound histotripsy, cryoablation, as well as a myriad of other surgical procedures. In each instance, a probe, drill, agitator, inserter (such as a radiation seed inserter), or other device is inserted in the hollow portion of the depth advancement marker needle to provide guided placement of that device during surgical procedures.

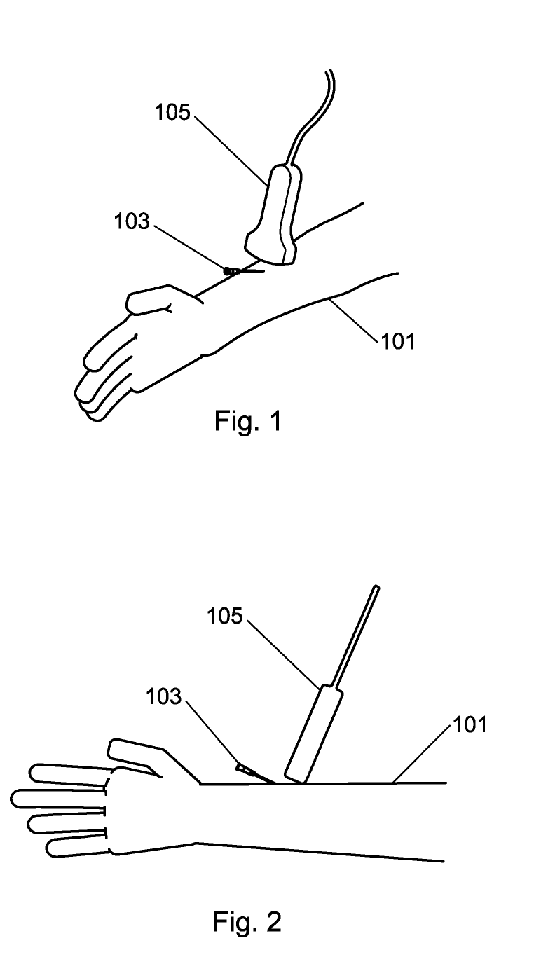

Turning now to the various figures provided, FIG. 1 depicts a perspective view of an ultrasonic transducer probe performing short axis imaging of an image guided injection. In this example, a patient's arm 101 can be seen with a medical needle 103 placed therein and an ultrasonic transducer 105 being employed to provide a short axis image of the procedure. The resulting short axis image will provide a view of the needle in cross section, showing essentially a small circle with the associated feature for depth measurement. FIG. 2 is a plan view of the imaging procedure of FIG. 1 that further shows the placement of the transducer to achieve short axis imaging. While the transducer depicted is an ultrasonic transducer, other imaging systems, such as, for example, computed tomography (CT) or magnetic resonance imaging (MRI) systems, may also be employed.

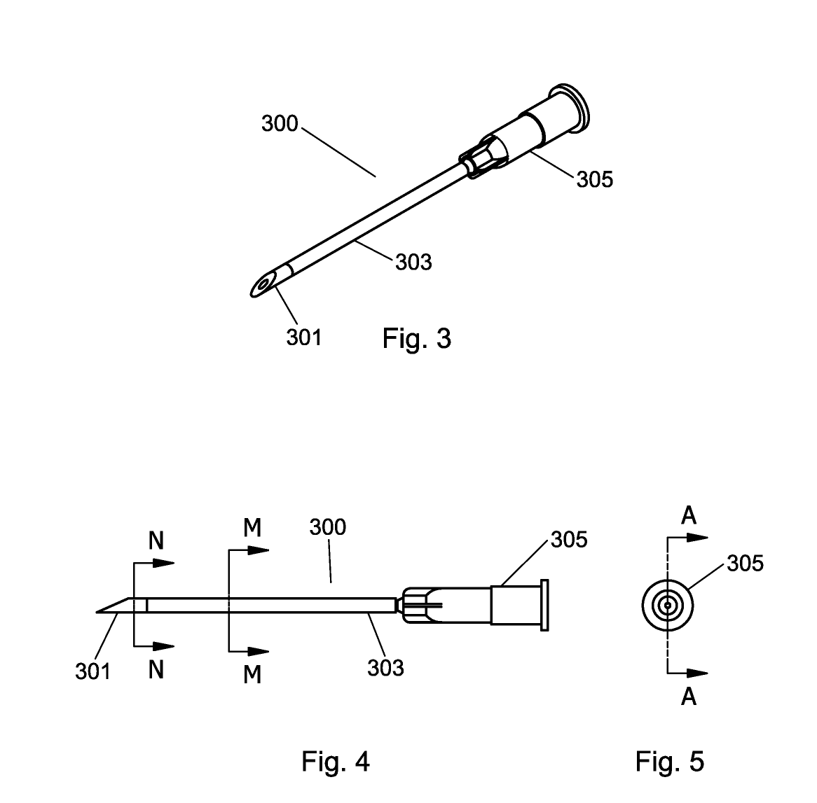

A first embodiment of the depth advancement marker needle is depicted in FIGS. 3-8. In this embodiment, the tip and the shaft of the needle are of different diameters such that they appear as different size circles or dots in a short axis image, thus allowing the practitioner to determine depth of the needle as it relates to the image “slice” taken. FIG. 3 depicts a perspective view of a first embodiment of a depth advancement marker needle 300. In FIG. 3, the tip 301 is of a larger diameter than the shaft 701 of the needle (see cross sectional view in FIG. 8). To ensure uniform diameter of the overall exterior of the needle, in some embodiments of the present invention a biocompatible shaft coating 303 is applied such that the diameter of the tip 301 is generally the same as the diameter of the shaft coating 303. The shaft coating, however, has a different imaging property than the tip 301 and shaft 701 (which may, in some embodiments of the present invention be made from the same material, such as, for example, stainless steel). Preferably, the shaft coating is transparent or opaque to an imaging system such that only the tip diameter and the shaft diameter can be seen, allowing for the change in diameter between tip and shaft to be an indicator of depth. A fastener 305 is also attached to the needle itself to allow connection to a device such as, for example, a syringe. Fastener 305 may include, for example, a LUER-LOCK® or a LUER-SLIP® connector. LUER-LOCK® and LUER-SLIP® are registered trademarks of Becton Dickinson and Company of Franklin Lakes, N.J. In some embodiments of the present invention, the shaft may be of a larger diameter than the tip. The larger diameter section of the depth advancement marker needle may be formed by varying the draw and die treatment during fabrication, or may, in some embodiments of the present invention, be a separate larger piece that is attached to the smaller diameter section by way of friction fit, heat or cold treatment, welding, use of adhesives, and the like.

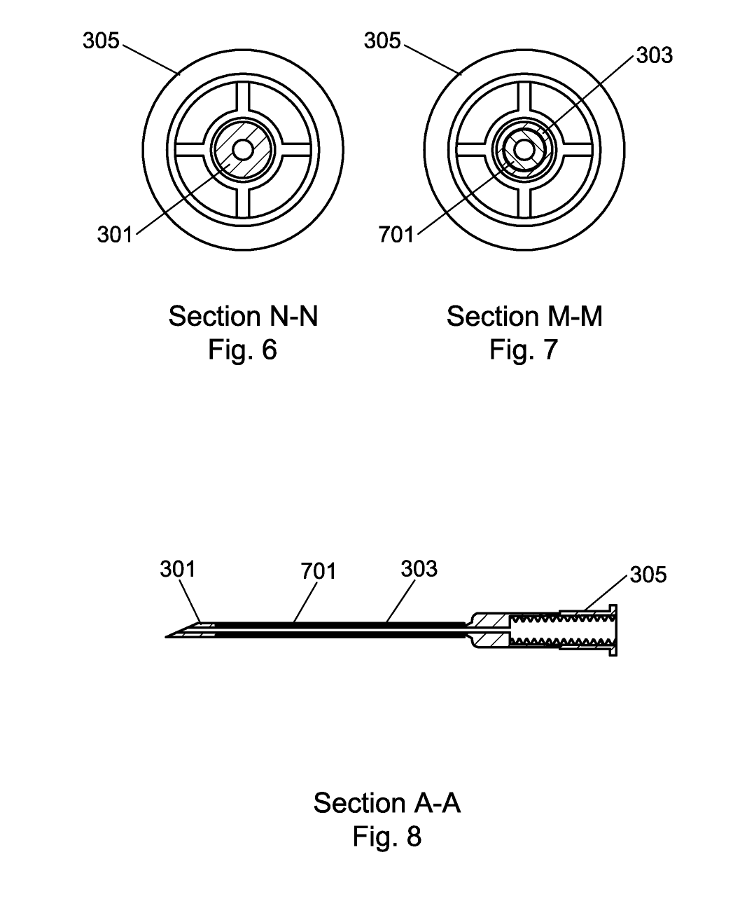

FIG. 4 is a plan view of a first embodiment of the depth advancement marker needle clearly showing the tip 301 and shaft coating 303 sections. FIG. 5 is an end view of a first embodiment of a depth advancement marker needle. FIG. 6 is a cross sectional view of the depth advancement marker needle taken along line N-N of FIG. 4 and showing the larger diameter tip 301. FIG. 7 is a cross sectional view of the depth advancement marker needle taken along line M-M of FIG. 4 and showing the smaller diameter shaft 701 and the shaft coating 303. FIG. 8 is a cross sectional view of the depth advancement marker needle taken along line A-A of FIG. 5 where the shaft coating 303 can be seen surrounding the smaller diameter shaft 701 such that the diameter of the tip 301 and the diameter of the shaft coating 303 are generally the same.

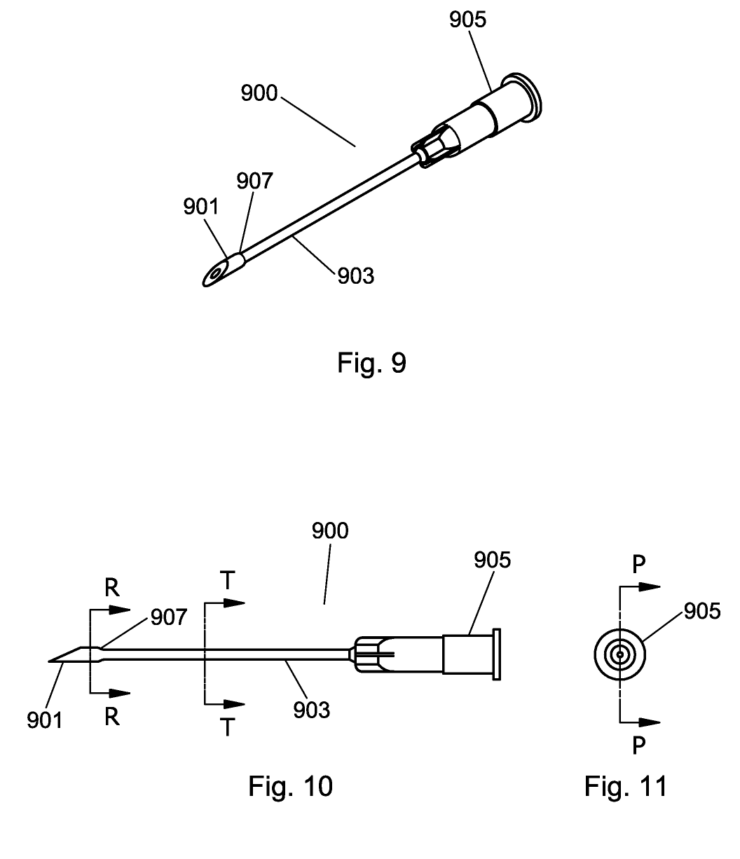

A second embodiment of the depth advancement marker needle is depicted in FIGS. 9-14. In this embodiment, the tip and the shaft of the needle are of different diameters such that they appear as different size circles or dots in a short axis image, thus allowing the practitioner to determine depth of the needle as it relates to the image “slice” taken. In this second embodiment, there is no shaft coating. FIG. 9 depicts a perspective view of a second embodiment of a depth advancement marker needle 900. In FIG. 9, the tip 901 is of a larger diameter than the shaft 903 of the needle. The tip 901 and the shaft 903 may, in some embodiments of the present invention be made from the same material, such as, for example, stainless steel. When imaged, the change in diameter between tip and shaft is an indicator of depth. A transition 907 between the tip 901 and the shaft 903 may be an angle, a curve, a bevel, or similar shape such that there is not an abrupt change in diameter between the tip and shaft that could tear tissue or create other maladies. A fastener 905 is also attached to the needle itself to allow connection to a device such as, for example, a syringe. Fastener 305 may include, for example, a LUER-LOCK® or a LUER-SLIP® connector. LUER-LOCK® and LUER-SLIP® are registered trademarks of Becton Dickinson and Company of Franklin Lakes, N.J. In some embodiments of the present invention, the shaft may be of a larger diameter than the tip. The larger diameter section of the depth advancement marker needle may be formed by varying the draw and die treatment during fabrication, or may, in some embodiments of the present invention, be a separate larger piece that is attached to the smaller diameter section by way of friction fit, heat or cold treatment, welding, use of adhesives, and the like.

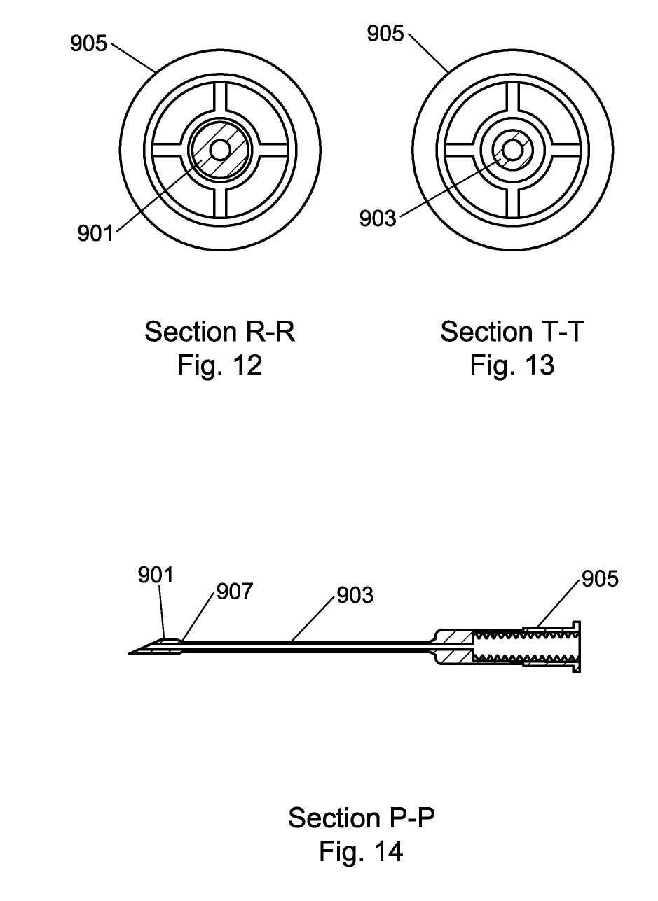

FIG. 10 is a plan view of a second embodiment of the depth advancement marker needle clearly showing the tip 901 and shaft 903 sections as well as the transition 907. FIG. 11 is an end view of a second embodiment of a depth advancement marker needle. FIG. 12 is a cross sectional view of the depth advancement marker needle taken along line R-R of FIG. 10 and showing the larger diameter tip 901. FIG. 13 is a cross sectional view of the depth advancement marker needle taken along line T-T of FIG. 10 and showing the smaller diameter shaft 903. FIG. 14 is a cross sectional view of the depth advancement marker needle taken along line P-P of FIG. 11 where the smaller diameter shaft 303 can be seen along with the larger diameter tip 901 and the transition 907.

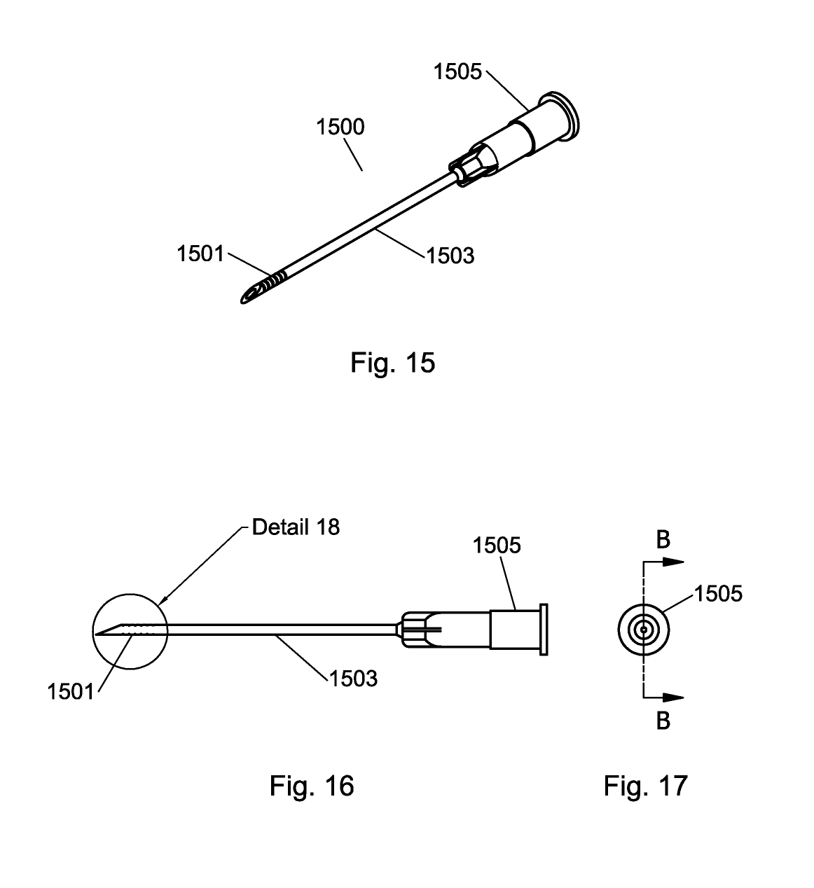

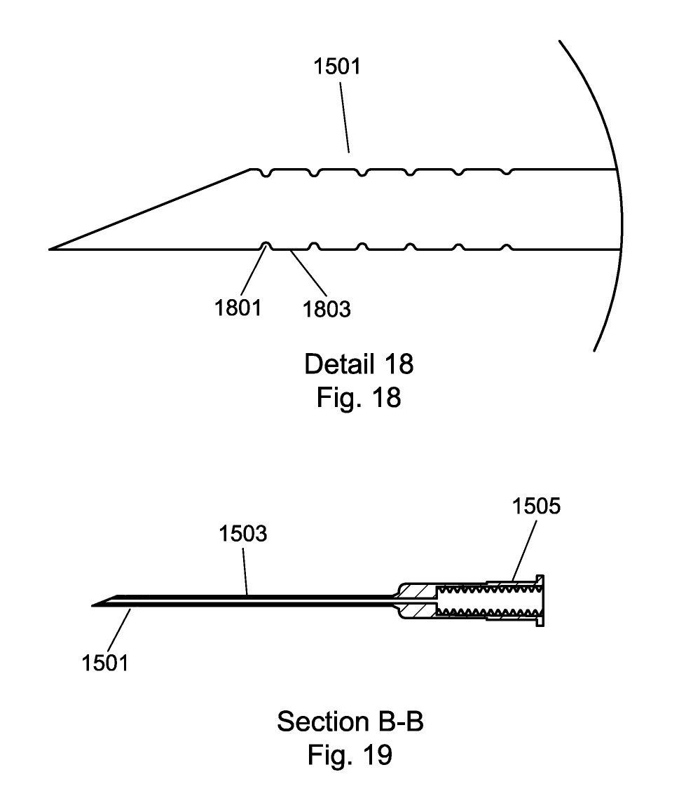

FIG. 15 is a perspective view of a third embodiment of a depth advancement marker needle where a series of grooves and islands are employed on the exterior of the needle to indicate depth in short axis imaging applications. In some embodiments of the present invention, the grooves are progressively larger or smaller to provide additional depth information. The depth advancement marker needle 1500 has a grooved tip 1501 such as a progressively grooved tip as shown in FIG. 18. A shaft 1503 is then connected to a fastener 1505 to allow connection to a device such as, for example, a syringe. Fastener 1505 may include, for example, a LUER-LOCK® or a LUER-SLIP® connector. LUER-LOCK® and LUER-SLIP® are registered trademarks of Becton Dickinson and Company of Franklin Lakes, N.J. The shaft 1503 and the tip 1501 may be made from a metal, such as, for example, stainless steel.

FIG. 16 is a plan view of a third embodiment of the depth advancement marker needle showing the grooved tip 1501 and the shaft 1503 connected to a fastener 1505. FIG. 17 is an end view of a third embodiment of a depth advancement marker needle. FIG. 18 is a close-up detail view of the depth advancement marker needle of FIG. 16. In FIG. 18, grooves 1801 and islands 1803 can be seen. Islands 1803 are essentially the area between the grooves 1801, and they may be of uniform size, or may, in some embodiments of the present invention, vary in size. The grooves 1801 may, in some embodiments of the present invention, vary in depth, getting progressively smaller or larger along the tip 1501. This creates varying diameter circles in a short axis image, thus providing further depth information. The islands 1803 may, in some embodiments of the present invention, vary in height or geometry, getting progressively smaller or larger along the tip 1501. In some embodiments of the present invention, the depth advancement marker needle is used with measurement software to measure the variation in diameter along the shaft or tip of the needle and translate that diameter change into depth of penetration information. FIG. 19 is a cross sectional view of the depth advancement marker needle taken along line B-B of FIG. 17. The grooved tip 1501 can be seen along with the shaft 1503 and the connector 1505.

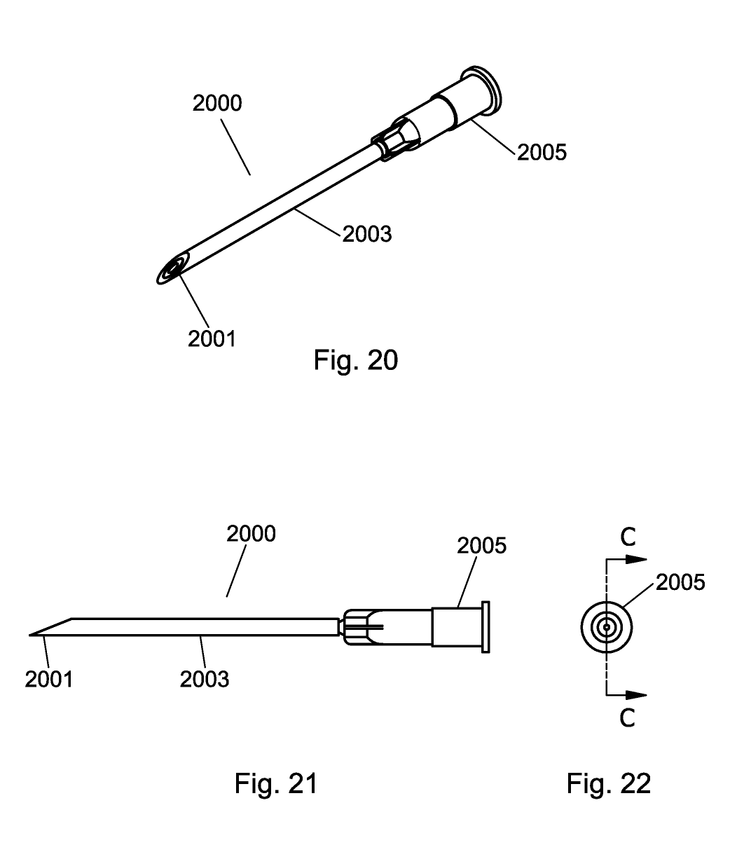

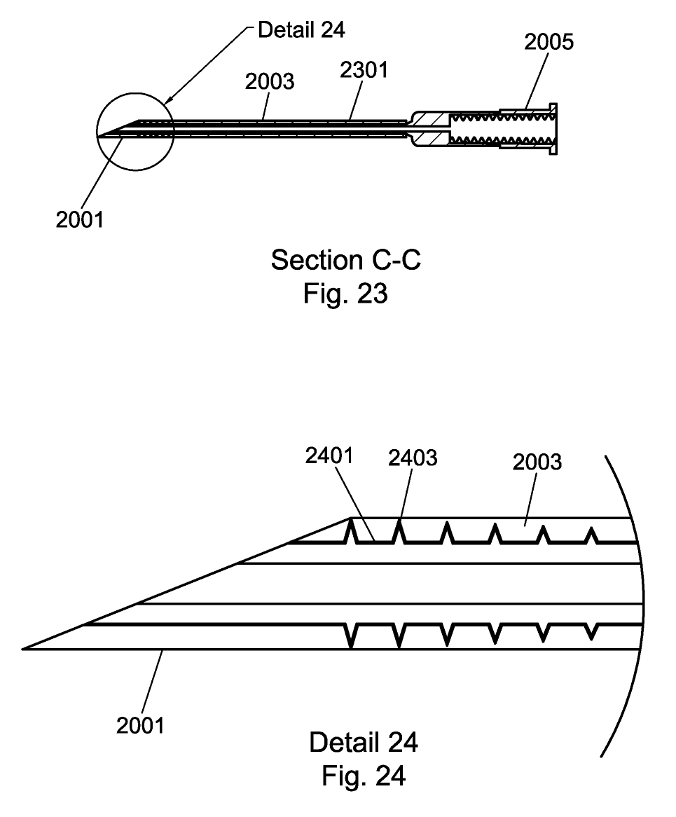

FIG. 20 is a perspective view of a fourth embodiment of a depth advancement marker needle where a series of protrusions and islands are employed on the needle to indicate depth in short axis imaging applications. In some embodiments of the present invention, the protrusions are progressively larger or smaller to provide additional depth information. The depth advancement marker needle 2000 has a tip 2001 such as the tip as shown in FIG. 24. A shaft coating 2003 covers the shaft 2301 (see FIG. 23) and the shaft 2301 is then connected to a fastener 2005 to allow connection to a device such as, for example, a syringe. Fastener 2005 may include, for example, a LUER-LOCK® or a LUER-SLIP, connector. LUER-LOCK® and LUER-SLIP® are registered trademarks of Becton Dickinson and Company of Franklin Lakes, N.J. The shaft 2301 and the tip 2001 may be made from a metal, such as, for example, stainless steel). To ensure uniform diameter of the overall exterior of the needle, in some embodiments of the present invention a biocompatible shaft coating 2003 is applied such that the diameter of the tip 2001 is generally the same as the diameter of the shaft coating 2003. The shaft coating, however, has a different imaging property than the tip 2001 and shaft 2301 (which may, in some embodiments of the present invention be made from the same material, such as, for example, stainless steel). Preferably, the shaft coating 2003 is transparent or opaque to an imaging system such that only the tip diameter and the shaft diameter can be seen, allowing for the change in diameter between the peaks and valleys of the protrusions to be an indicator of depth.

FIG. 21 is a plan view of a fourth embodiment of the depth advancement marker needle showing the tip 2001 and the shaft coating 2003 connected to a fastener 2005. FIG. 22 is an end view of a fourth embodiment of a depth advancement marker needle. FIG. 23 is a cross sectional view of the depth advancement marker needle taken along line C-C of FIG. 22. The tip 2001 can be seen along with the shaft 2301 and the shaft coating 2003 as well as the connector 2005.

FIG. 24 is a close-up detail view of the depth advancement marker needle of FIG. 23. In FIG. 24, protrusions 2403 and valleys 2401 can be seen. Valleys 2401 are essentially the area between the protrusions 2403, and they may be of uniform size, or may, in some embodiments of the present invention, vary in size. The protrusions 2403 may, in some embodiments of the present invention, vary in height or geometry, getting progressively smaller or larger along the tip 2001. This creates varying diameter circles in a short axis image, thus providing further depth information. The valleys 2401 may, in some embodiments of the present invention, vary in height or geometry, getting progressively smaller or larger along the tip 2001. In some embodiments of the present invention, the depth advancement marker needle is used with measurement software to measure the variation in diameter along the shaft or tip of the needle and translate that diameter change into depth of penetration information.

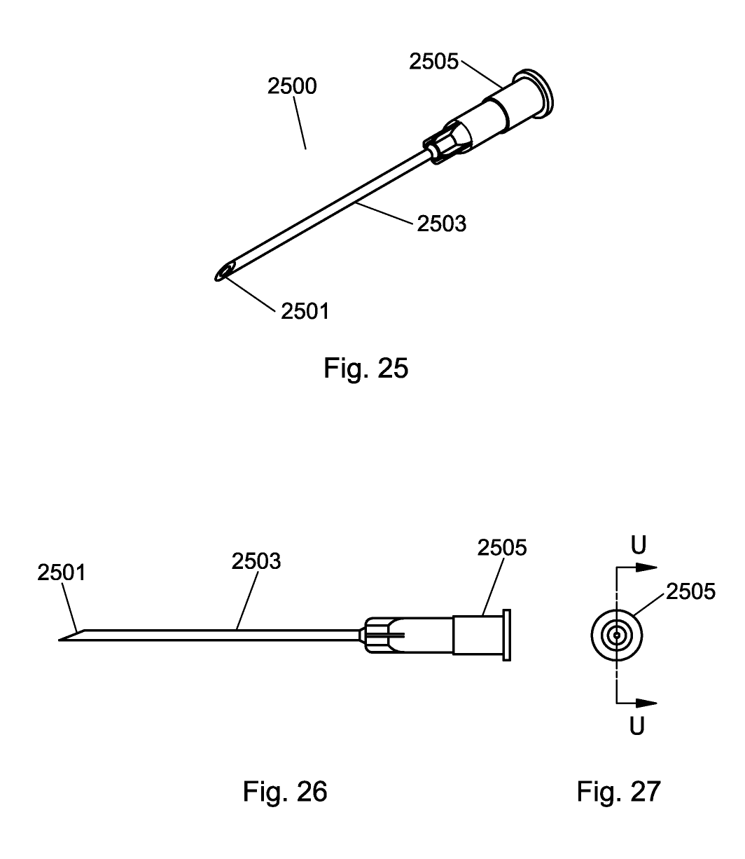

FIG. 25 is a perspective view of a fifth embodiment of a depth advancement marker needle where a series of grooves and islands are employed on the interior of the needle to indicate depth in short axis imaging applications. In some embodiments of the present invention, the grooves are progressively larger or smaller to provide additional depth information. The depth advancement marker needle 2500) has a tip 2501 such as an interior grooved tip as shown in FIG. 29. A shaft 2503 is then connected to a fastener 2505 to allow connection to a device such as, for example, a syringe. Fastener 2505 may include, for example, a LUER-LOCK® or a LUER-SLIP® connector. LUER-LOCK® and LUER-SLIP® are registered trademarks of Becton Dickinson and Company of Franklin Lakes, N.J. The shaft 2503 and the tip 2501 may be made from a metal, such as, for example, stainless steel.

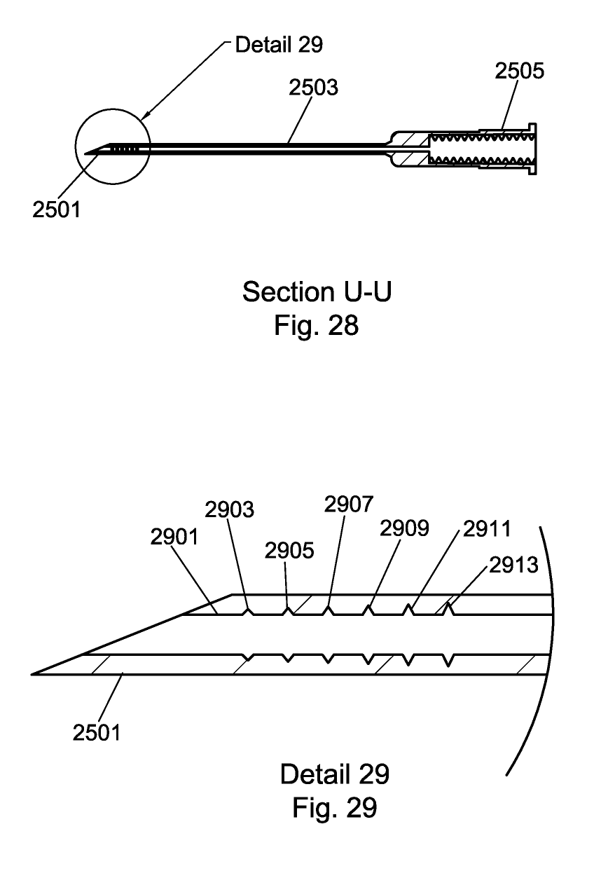

FIG. 26 is a plan view of a fifth embodiment of the depth advancement marker needle showing the interior grooved tip 2501 and the shaft 2503 connected to a fastener 2505. FIG. 27 is an end view of a fifth embodiment of a depth advancement marker needle. FIG. 28 is a cross sectional view of the depth advancement marker needle taken along line U-U of FIG. 27. The interior grooved tip 2501 can be seen along with the shaft 2503 and the connector 2505.

FIG. 29 is a close-up detail view of the depth advancement marker needle of FIG. 28. In FIG. 29, an inner wall 2901 is shown with a first groove 2903, a second groove 2905, a third groove 2907, a fourth groove 2909, a fifth groove 2911 and a sixth groove 2913. There may be more or fewer grooves in various embodiments of the present invention. The grooves may, in some embodiments of the present invention, vary in depth, getting progressively smaller or larger along the tip 2501. This creates varying diameter circles in a short axis image, thus providing further depth information. In some embodiments of the present invention, the depth advancement marker needle is used with measurement software to measure the variation in diameter along the shaft or tip of the needle and translate that diameter change into depth of penetration information.

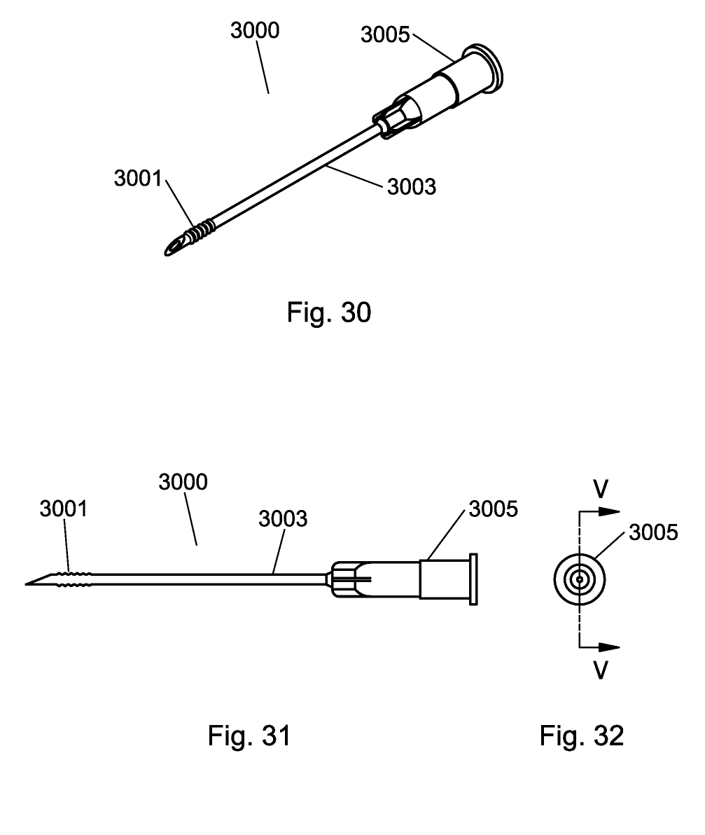

FIG. 30 is a perspective view of a sixth embodiment of a depth advancement marker needle where a series of protrusions and islands are employed on the exterior of the needle to indicate depth in short axis imaging applications. In some embodiments of the present invention, the protrusions are progressively larger or smaller to provide additional depth information. The depth advancement marker needle 3000 has a tip 3001 such as a progressive protrusion tip as shown in FIG. 34. A shaft 3003 is then connected to a fastener 3005 to allow connection to a device such as, for example, a syringe. Fastener 3005 may include, for example, a LUER-LOCK® or a LUER-SLIP® connector. LUER-LOCK® and LUER-SLIP® are registered trademarks of Becton Dickinson and Company of Franklin Lakes, N.J. The shaft 3003 and the tip 3001 may be made from a metal, such as, for example, stainless steel.

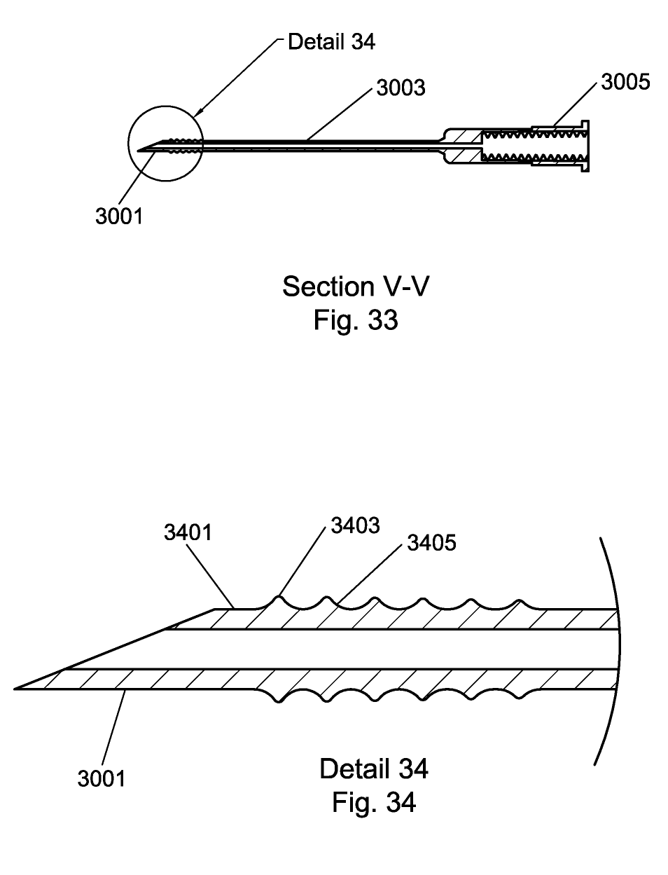

FIG. 31 is a plan view of a sixth embodiment of the depth advancement marker needle showing the tip 3001 and the shaft 3003 connected to a fastener 3005. FIG. 32 is an end view of a sixth embodiment of a depth advancement marker needle. FIG. 33 is a cross sectional view of the depth advancement marker needle taken along line V-V of FIG. 32. The protrusion tip 3001 can be seen along with the shaft 3003 and the connector 3005.

FIG. 34 is a close-up detail view of the depth advancement marker needle of FIG. 33. In FIG. 34, protrusions 3403 and islands 3405 can be seen. Islands 3405 are essentially the area between the protrusions 3403, and they may be of uniform size, or may, in some embodiments of the present invention, vary in size. The protrusions 3403 may, in some embodiments of the present invention, vary in height or other geometry, getting progressively smaller or larger along the tip 3001. The protrusions may have a taper, bevel, curve, or other transitional feature to prevent a discontinuity that could result in tissue damage or the like. In some embodiments of the present invention, the protrusions may be coated to create a smoother exterior surface of the needle. The protrusions create varying diameter circles in a short axis image, thus providing further depth information. In some embodiments of the present invention, the depth advancement marker needle is used with measurement software to measure the variation in diameter along the shaft or tip of the needle and translate that diameter change into depth of penetration information

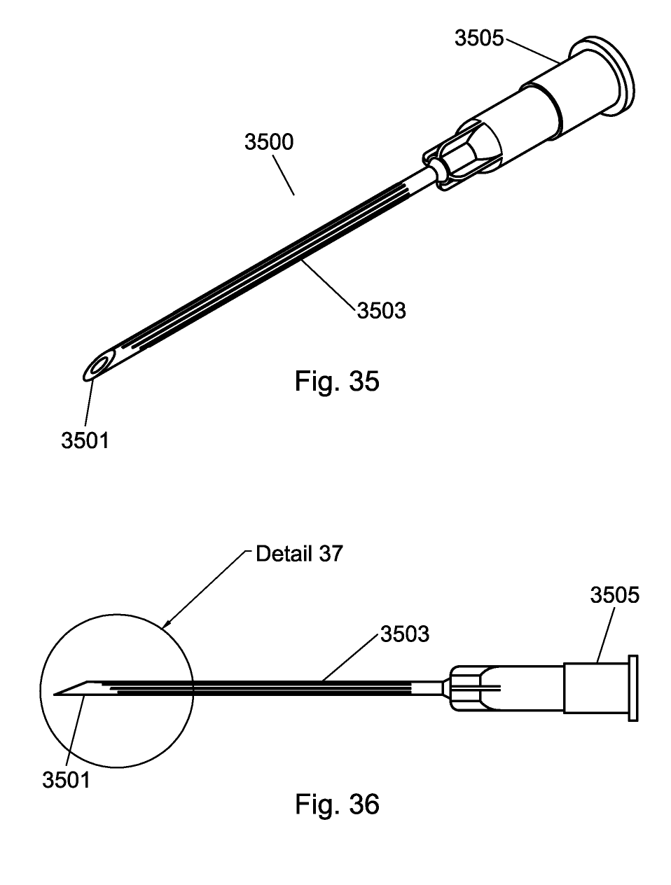

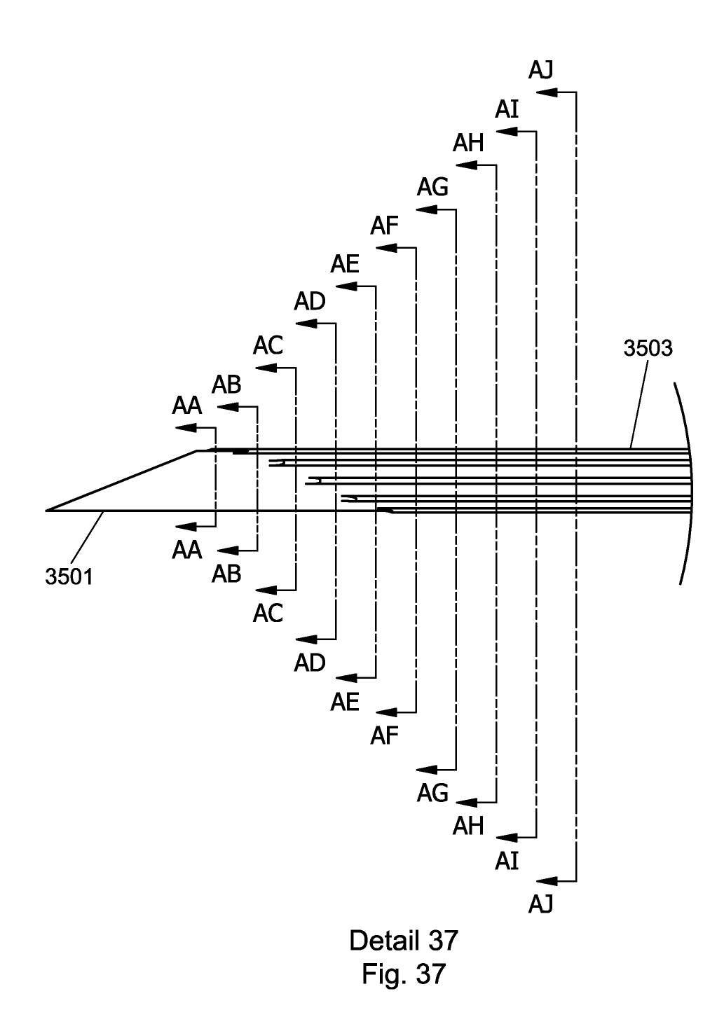

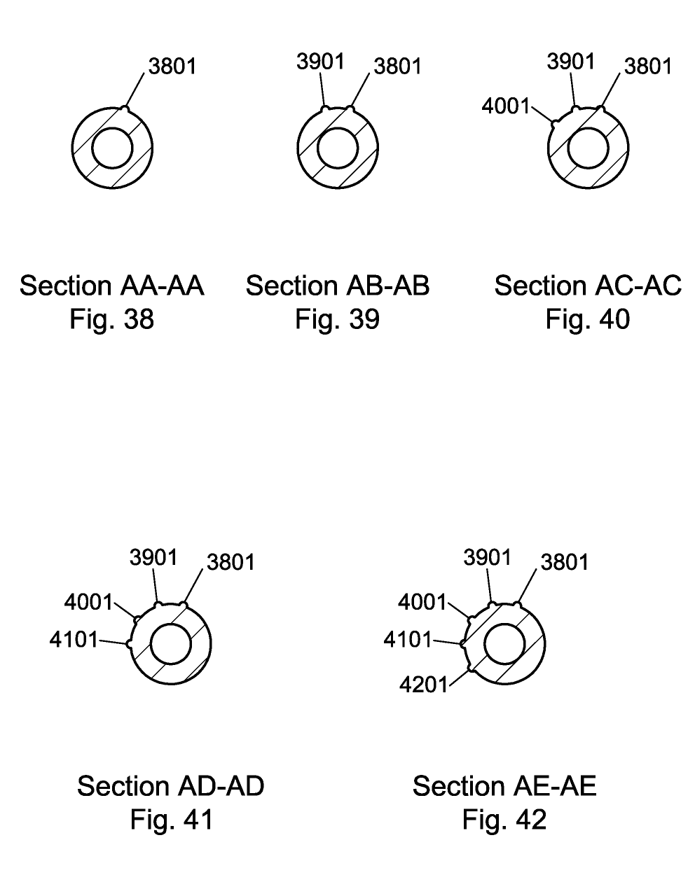

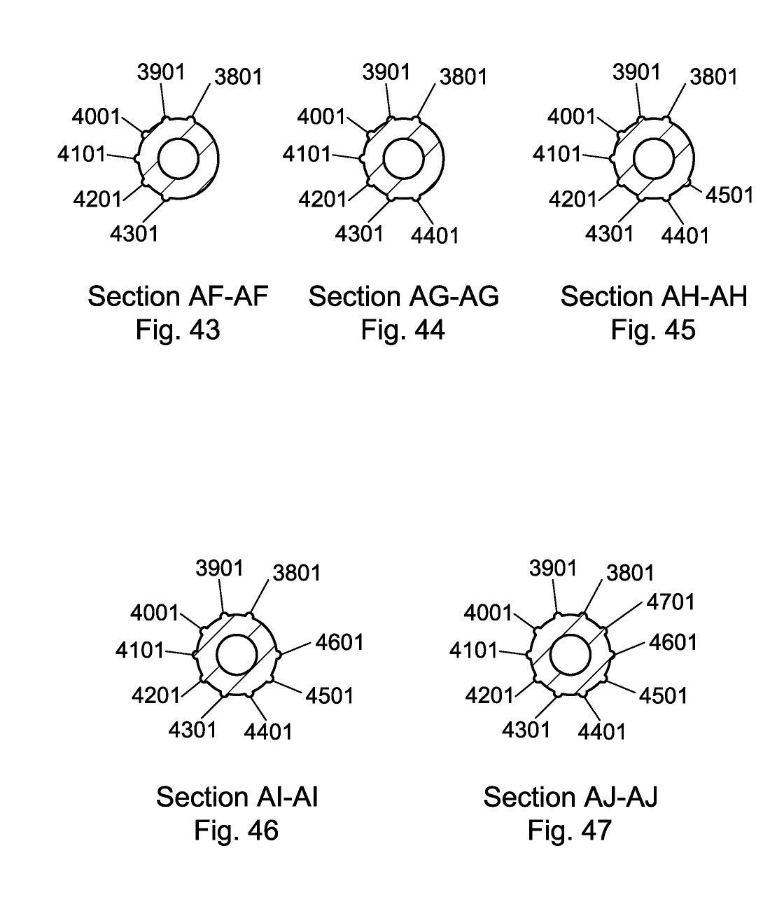

Turning now to FIG. 35 and the accompanying FIGS. 36-47, a seventh embodiment of a depth advancement marker needle is depicted and shown in perspective view in FIG. 35. The depth advancement marker needle 3500 has a tip 3501, a shaft 3503 and a fastener 3505. The shaft 3503 is connected to the fastener 3505 to allow connection to a device such as, for example, a syringe. Fastener 3505 may include, for example, a LUER-LOCK® or a LUER-SLIP®connector. LUER-LOCK® and LUER-SLIP® are registered trademarks of Becton Dickinson and Company of Franklin Lakes, N.J. The shaft 3503 and the tip 3501 may be made from a metal, such as, for example, stainless steel. As will be seen in later drawings and described further herein, the shall 3503 has indicators provided thereupon. The indicators are features that protrude or are recessed into the shaft 3503 and are generally parallel with the long axis of the shaft 3503. The indicators may be made from the same material as the shaft 3503, or in some embodiments of the present invention, may be made from a different material than that of the shaft provided it has the ability to be imaged. The indicators are each a different length so as to provide an indication of the depth of the needle. In one embodiment of the present invention, the indicators are staggered such that they are sequentially longer or shorter than the adjacent indicator. When imaged, this provides a series of marks around the periphery of the needle shaft cross section where the number of marks correspond to the depth of penetration of the needle. A scale convenient to the specific application of the needle may be used to correlate the number of marks with a depth measurement. FIG. 36 is a plan view of a seventh embodiment of the depth advancement marker needle that further depicts the indicators showing the varying lengths of each. FIG. 37 is a close-up detail view of the depth advancement marker needle of FIG. 36. The varying length indicators can be seen along the shaft 3503. The indicators may protrude from or be recessed in the shaft 3503. At a given depth, a certain number of indicators will be visible around the periphery of the image as seen in a short axis image. FIGS. 38-47 depict cross sectional views of the depth advancement marker needle similar to how a short axis image of the depth advancement marker needle would appear. As the needle progresses deeper into its environment, additional indicators will be seen. In the example depicted in the drawings, 10 indicators are employed. More or fewer indicators may also be used in various embodiments of the present invention. FIG. 38 is a cross sectional view of the depth advancement marker needle taken along line AA-AA of FIG. 37. FIG. 39 is a cross sectional view of the depth advancement marker needle taken along line AB-AB of FIG. 37. FIG. 40 is a cross sectional view of the depth advancement marker needle taken along line AC-AC of FIG. 37. FIG. 41 is a cross sectional view of the depth advancement marker needle taken along line AD-AD of FIG. 37. FIG. 42 is a cross sectional view of the depth advancement marker needle taken along line AE-AE of FIG. 37. FIG. 43 is a cross sectional view of the depth advancement marker needle taken along line AF-AF of FIG. 37. FIG. 44 is a cross sectional view of the depth advancement marker needle taken along line AG-AG of FIG. 37. FIG. 45 is a cross sectional view of the depth advancement marker needle taken along line AH-AH of FIG. 37. FIG. 46 is a cross sectional view of the depth advancement marker needle taken along line AI-AI of FIG. 37, and FIG. 47 is a cross sectional view of the depth advancement marker needle taken along line AJ-AJ of FIG. 37.

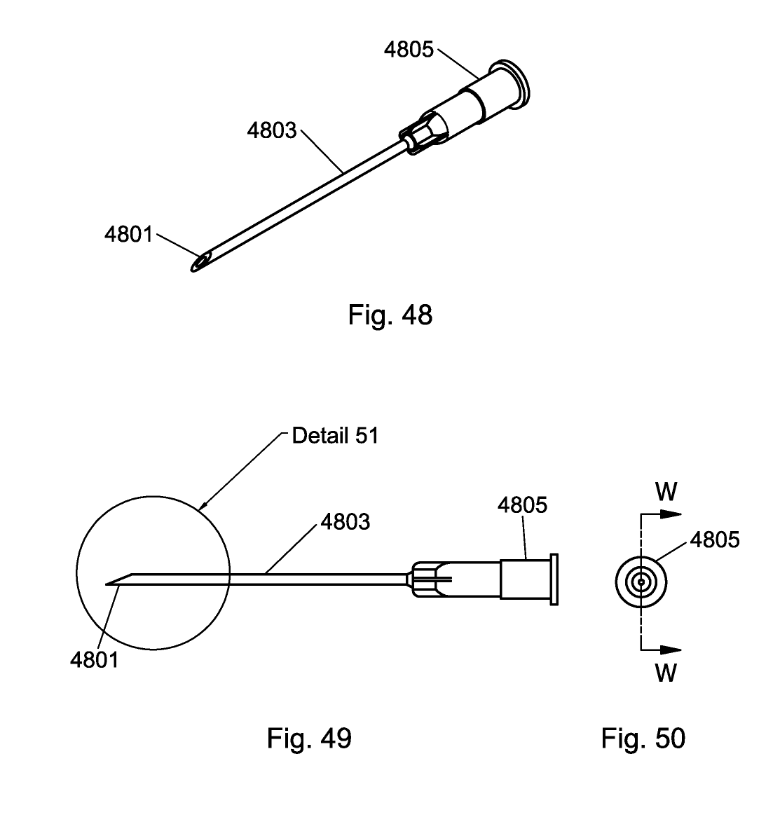

Turning now to FIG. 48 and the accompanying FIGS. 49-62, an eighth embodiment of a depth advancement marker needle is depicted and shown in perspective view in FIG. 49. The depth advancement marker needle has a tip 4801, a shaft 4803 and a fastener 4805. The shaft 4803 is connected to the fastener 4805 to allow connection to a device such as, for example, a syringe. Fastener 4805 may include, for example, a LUER-LOCK® or a LUER-SLIP® connector. LUER-LOCK® and LUER-SLIP® are registered trademarks of Becton Dickinson and Company of Franklin Lakes, N.J. The shaft 4803 and the tip 4801 may be made from a metal, such as, for example, stainless steel. As will be seen in later drawings and described further herein, the inner wall of the needle shaft has markings similar to the configuration of markings described by way of FIGS. 35-47. The indicators are features that protrude or are recessed into the inner wall of the shaft 4803 and are generally parallel with the long axis of the shaft 4803. The indicators may be made from the same material as the shaft 4803, or in some embodiments of the present invention, may be made from a different material than that of the shaft provided it has the ability to be imaged. The indicators are each a different length so as to provide an indication of the depth of the needle. In one embodiment of the present invention, the indicators are staggered such that they are sequentially longer or shorter than the adjacent indicator. When imaged, this provides a series of marks around the inner periphery of the needle shaft cross section where the number of marks correspond to the depth of penetration of the needle. A scale convenient to the specific application of the needle may be used to correlate the number of marks with a depth measurement. FIG. 49 is a plan view of an eighth embodiment of the depth advancement marker needle that further depicts the indicators showing the varying lengths of each. FIG. 50 is an end view of an eighth embodiment of a depth advancement marker needle;

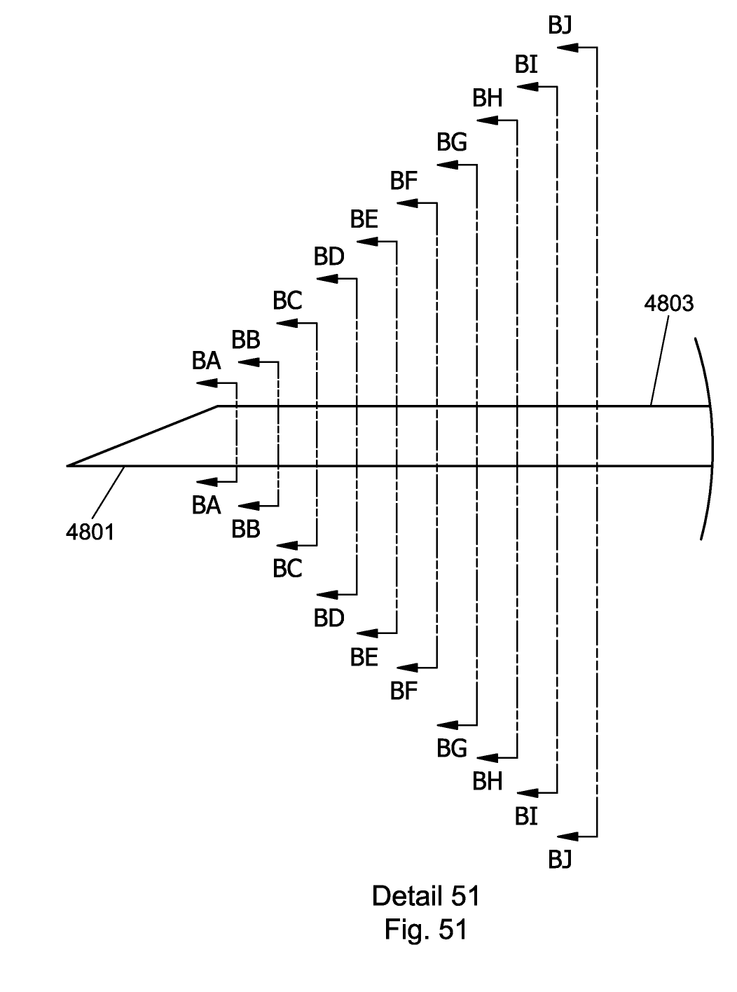

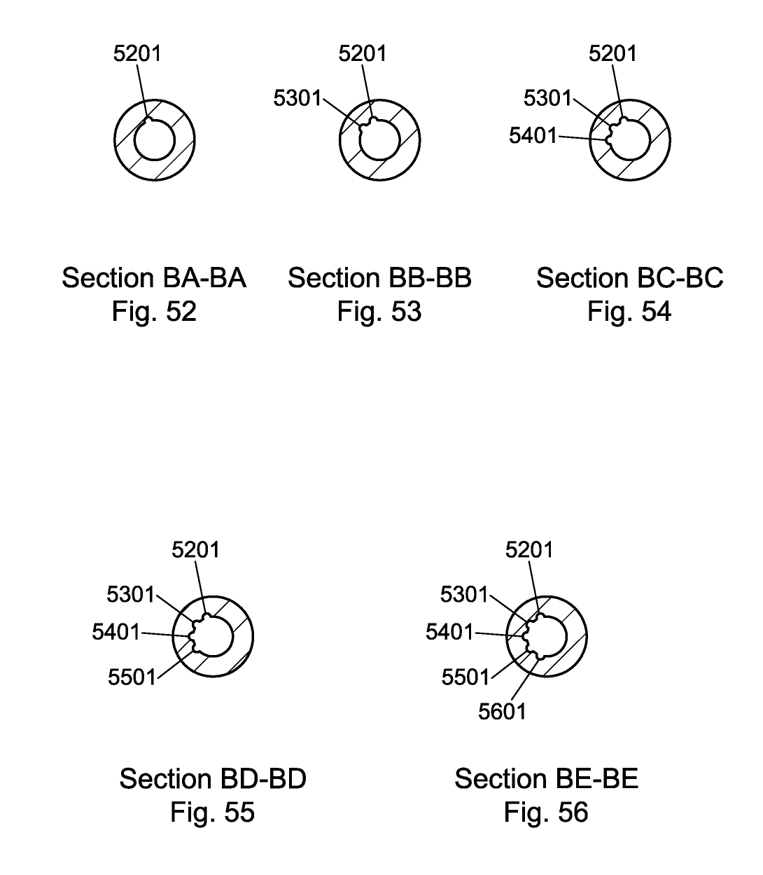

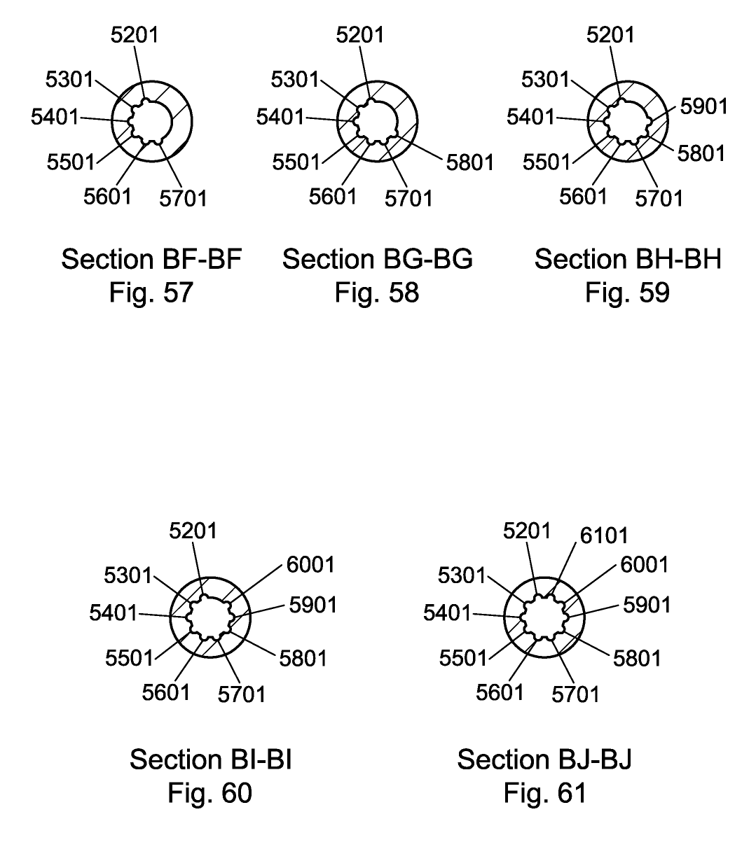

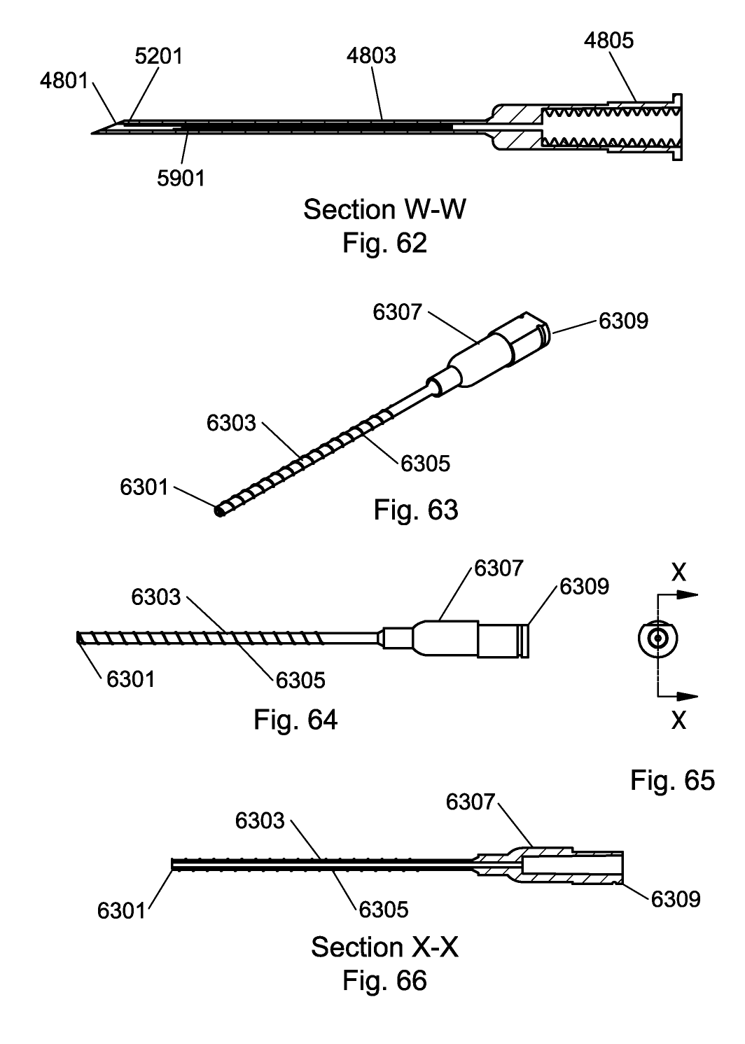

FIG. 51 is a close-up detail view of the depth advancement marker needle of FIG. 49. The varying length indicators can be seen within the shaft 4803. The indicators may protrude from or be recessed in the inner wall of the shaft 4803. At a given depth, a certain number of indicators will be visible around the inner periphery of the image as seen in a short axis image. FIGS. 52-61 depict cross sectional views of the depth advancement marker needle similar to how a short axis image of the depth advancement marker needle would appear. As the needle progresses deeper into its environment, additional indicators will be seen. In the example depicted in the drawings, 10 indicators are employed. More or fewer indicators may also be used in various embodiments of the present invention. FIG. 52 is a cross sectional view of the depth advancement marker needle taken along line BA-BA of FIG. 51. FIG. 53 is a cross sectional view of the depth advancement marker needle taken along line BB-BB of FIG. 51. FIG. 54 is a cross sectional view of the depth advancement marker needle taken along line BC-BC of FIG. 51. FIG. 55 is a cross sectional view of the depth advancement marker needle taken along line BD-BD of FIG. 51. FIG. 56 is a cross sectional view of the depth advancement marker needle taken along line BE-BE of FIG. 51. FIG. 57 is a cross sectional view of the depth advancement marker needle taken along line BF-BF of FIG. 51. FIG. 58 is a cross sectional view of the depth advancement marker needle taken along line BG-BG of FIG. 51. FIG. 59 is a cross sectional view of the depth advancement marker needle taken along line BH-BH of FIG. 51. FIG. 60 is a cross sectional view of the depth advancement marker needle taken along line BI-BI of FIG. 51. FIG. 61 is a cross sectional view of the depth advancement marker needle taken along line BJ-BJ of FIG. 51, and FIG. 62 is a cross sectional view of the depth advancement marker needle taken along line W-W of FIG. 50.

The various markers and indicators used for depth measurement as described herein, while portrayed either on the surface of the needle or the inner wall of the needle, may also be embedded in the needle wall, layered, coated, spray applied, placed through the needle wall, or arranged in some other configuration that would provide depth indication in a short axis imaging environment. The placement of the markers, and in that regard the size and geometry of the markers, may vary, and these various modifications, additions, and embodiments are to be considered within the spirit and broad scope of the present invention as described and envisioned herein.

FIG. 63 is a perspective view of a ninth embodiment of a depth advancement marker needle. While the various configurations and embodiments of depth advancement marker needles described and envisioned herein may, in some embodiments of the present invention, rely on incision by way of a sharpened or beveled tip, the depth advancement marker needles may also be advanced in penetration depth by way of other means, such as, for example, a screw type arrangement. FIGS. 62-66 depict such an arrangement, however, screw or other mechanical means for advancement may also be adapted to the marker needle embodiments that have been previously described or envisioned herein. FIG. 63 depicts a depth advancement marker needle having a tip 6301, a shaft 6303, and a screw marker 6305. The screw marker 6305 comprises a helical protrusion or a protrusion capable of advancing the needle mechanically through the application of an external force, such as, for example, a rotational force. The screw marker 6305 correlates the number of turns of the needle to depth, and may, in some embodiments of the present invention, provide further depth information by way of imaging. In some embodiments of the present invention, a counter mechanism such as a sleeve may be employed that advances and retracts to indicate or specify depth. In some embodiments of the present invention there may be markers on the coupler 6309 or elsewhere on the depth advancement marker needle to indicate or specify depth independent of counting the number of turns made by the needle. The fastener 6307 connects the needle to an external fixture such as a syringe or the like. A coupler 6309 provides the ability to connect the depth advancement marker needle to an external tool such as a surgical drill, surgical hand tool, manipulative extension, grip, handle, lever, gear, engagement device, or the like. A threaded sleeve may also be employed in some embodiments of the present invention to facilitate depth advancement of the marker needle. For complete understanding of this embodiment of the depth advancement marker needle. FIG. 64 is a plan view, FIG. 65 is an end view, and FIG. 66 is a cross sectional view of the depth advancement marker needle taken along line X-X of FIG. 65.

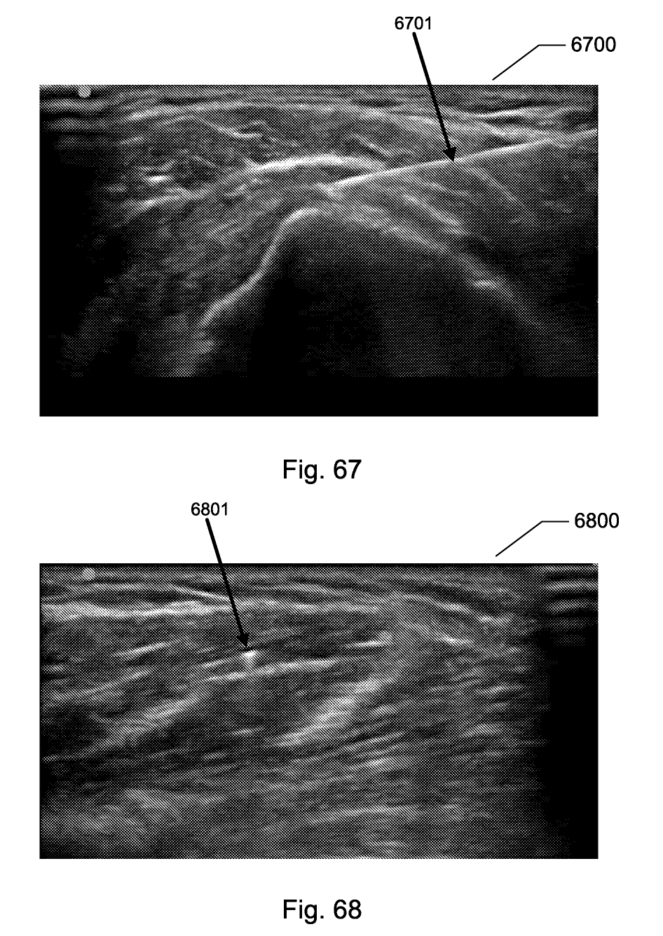

Lastly, FIGS. 67 and 68 depict actual images of an image guided needle injection in both the long axis and the short axis orientation. FIG. 67 is an ultrasound long axis image of an image guided injection procedure. As can be seen, the entire length of the medical needle can be seen and measured, thus determination of depth and needle placement is not a problem for the practitioner. FIG. 68 is an ultrasound short axis image of an image guided injection procedure. Short axis imaging is often times a preferential imaging orientation in some instances except for the fact that proper depth measurement is difficult to determine. As can be seen in FIG. 68, the medical needle appears as a dot or a small circle regardless of the depth of penetration. This is problematic for the practitioner, the clinician, the technician, and the patient. The present invention, and the various embodiments described and envisioned herein overcome this problem and provide a depth measurement device and method that has heretofore been unknown.

To use the depth advancement marker needle for image guided procedures, the marker needle is placed into the patient during an image guided procedure such as sonography. One method of determining depth of penetration of a depth advancement marker needle during an image guided procedure where the depth advancement marker needle is imaged with a short axis orientation involves the following steps that can be undertaken either by a medical practitioner or by a machine where the steps are undertaken in whole or in part using software that is resident on a computer that is operatively connected to an imaging machine. The machine may be, for example, an ultrasound imaging machine (sonograph). An exemplary method comprises the steps of locating the short axis image of the depth advancement marker needle. This can be done by visualizing the rendered image either on a computer screen, on a paper plot, or using, for example, image processing software. Once the short axis image of the depth advancement marker needle is located, the visual indicator feature of the depth advancement marker needle is viewed either on a computer screen, on a paper plot, or using, for example, image processing software. As previously described, each embodiment of the depth advancement marker needle described or envisioned herein comprises a visual indicator feature that can be translated into a depth measurement. That visual indicator feature may be, for example, a different diameter shaft or a feature on the shaft of the depth advancement marker needle that makes a change in diameter or size evident upon imaging. The different size can then be correlated to depth of penetration of the marker needle. In some embodiments described herein, the visual indicator feature may be a series of markings that are either present in, or absent from, a short axis image of the marker needle. The number of markings evident in a given image is then correlated to depth of penetration of the marker needle. Once the attribute (for example, size or quantity) of the visual indicator feature is quantified, this quantified attribute is then translated into a depth of penetration measurement. For example, each marking that is visible in a short axis image may correlate to a unit of measure, such as millimeters, centimeters, or the like. In a similar manner, a specific size cross section may indicate a specific depth of penetration.

It is, therefore, apparent that there has been provided, in accordance with the various objects of the present invention, a Depth Advancement Marker Needle For Image Guided Procedures.

While the various objects of this invention have been described in conjunction with preferred embodiments thereof, it is evident that many alternatives, modifications, and variations will be apparent to those skilled in the art. Accordingly, it is intended to embrace all such alternatives, modifications and variations that fall within the spirit and broad scope of this specification, drawings, and claims provided herein.

What is claimed is:

1. A depth advancement marker needle for image guided procedures comprising:
a hollow metal wire having a shaft, a sharp end, and a fastening end; and
a feature that provides a visual indicator of depth of penetration while performing short axis imaging.

2. The depth advancement marker needle of claim 1, wherein the feature comprises a tip that is of a larger diameter than the diameter of the shaft of the depth advancement marker needle such that the tip appears as a larger image than the shaft during short axis imaging procedures.

3. The depth advancement marker needle of claim 2, further comprising a biocompatible shaft coating applied to the shaft such that the diameter of the tip is generally the same as the diameter of the biocompatible shaft coating and where the biocompatible shaft coating has a different imaging property than the tip and the shaft.

4. The depth advancement marker needle of claim 1, wherein the feature comprises a plurality of grooves that circumscribe the shaft of the depth advancement marker needle and wherein each consecutive groove is separated by an island that also circumscribes the shaft of the depth advancement marker needle.

5. The depth advancement marker needle of claim 4, wherein each groove varies in size relative to an adjacent groove such that each groove appears as a different size during short axis imaging procedures such that the imaged size of the groove correlates to depth of penetration of the depth advancement marker needle.

6. The depth advancement marker needle of claim 4, wherein each island varies in size relative to an adjacent island such that each island appears as a different size during short axis imaging procedures such that the imaged size of the island correlates to depth of penetration of the depth advancement marker needle.

7. The depth advancement marker needle of claim 4, further comprising a biocompatible shaft coating applied to the shaft such that the diameter of the shaft is generally uniform and where the biocompatible shaft coating has a different imaging property than the tip and the shaft.

8. The depth advancement marker needle of claim 1, wherein the feature comprises a plurality of protrusions that circumscribe the shaft of the depth advancement marker needle and wherein each consecutive protrusion is separated by a valley that also circumscribes the shaft of the depth advancement marker needle.

9. The depth advancement marker needle of claim 8, wherein each protrusion varies in size relative to an adjacent protrusion such that each protrusion appears as a different size during short axis imaging procedures such that the imaged size of the protrusion correlates to depth of penetration of the depth advancement marker needle.

10. The depth advancement marker needle of claim 8, wherein each valley varies in size relative to an adjacent valley such that each valley appears as a different size during short axis imaging procedures such that the imaged size of the valley correlates to depth of penetration of the depth advancement marker needle.

11. The depth advancement marker needle of claim 8, further comprising a biocompatible shaft coating applied to the shaft such that the diameter of the shaft is generally uniform and where the biocompatible shaft coating has a different imaging property than the tip and the shaft.

12. The depth advancement marker needle of claim 1, wherein the feature comprises a plurality of grooves on the interior of the shaft of the depth advancement marker needle and wherein each consecutive groove is separated by an island on the interior of the shaft of the depth advancement marker needle.

13. The depth advancement marker needle of claim 12, wherein each groove varies in size relative to an adjacent groove such that each groove appears as a different size during short axis imaging procedures such that the imaged size of the groove correlates to depth of penetration of the depth advancement marker needle.

14. The depth advancement marker needle of claim 12, wherein each island varies in size relative to an adjacent island such that each island appears as a different size during short axis imaging procedures such that the imaged size of the island correlates to depth of penetration of the depth advancement marker needle.

15. The depth advancement marker needle of claim 1, wherein the feature comprises a screw marker wherein the screw marker is a helical protrusion on the shaft of the depth advancement marker needle.

16. A depth advancement marker needle for image guided procedures comprising:
a hollow metal wire having a shaft with an interior and an exterior, a sharp end, and a fastening end; and
a series of indicators where each indicator is generally parallel with the long axis of the shaft;
wherein each indicator is staggered such that it is sequentially longer or shorter than an adjacent indicator to provide, during short axis imaging, a series of marks around an imaged periphery of a cross section of the shaft where the number of marks indicates depth of penetration of the depth advancement marker needle.

17. The depth advancement marker needle of claim 16, wherein the series of indicators protrude from the exterior of the shaft.

18. The depth advancement marker needle of claim 16, wherein the series of indicators are recessed with the exterior of the shaft.

19. The depth advancement marker needle of claim 16, wherein the series of indicators protrude from the interior of the shaft.

20. The depth advancement marker needle of claim 16, wherein the series of indicators are recessed with the interior of the shaft

21. A method of determining depth of penetration of a depth advancement marker needle during an image guided procedure where the depth advancement marker needle is imaged with a short axis orientation, the method comprising the steps of:
locating the short axis image of the depth advancement marker needle;
viewing the visual indicator feature of the depth advancement marker needle;
quantifying the attribute of the visual indicator feature; and
translating the quantified attribute of the visual indicator feature into a depth of penetration.
